(19) United States
(12) Patent Application Publication
(10) Pub. No.: US 2008/0075294 A1
(43) Pub. Date: Mar. 27, 2008

(54) LOW-NOISE MAIL HANDLING MACHINE

(75) Inventor: Fabien CHATTE, Nogent Sur Marne (FR)

Correspondence Address: SUGHRUE MION, PLLC, 2100 PENNSYLVANIA AVENUE, N.W., SUITE 800, WASHINGTON, DC, 20037, US

(73) Assignee: NEOPOST TECHNOLOGIES, French Limited Company, BAGNEUX (FR)

(21) Appl. No.: 11/857,851

(22) Filed: Sep. 19, 2007

(30) Foreign Application Priority Data
Sep. 21, 2006 (FR) 0653855

(51) Int. Cl. A61F 11/06

(52) U.S. Cl. 381/71.2

(57) ABSTRACT

In a mail handling machine comprising a succession of modules disposed along a conveyor path for conveying mail items from a feed module for feeding in mail items to a collection module for collecting mail items that have been franked by passing through a franking module for printing postage imprints on said mail items, there is provided at least one sound acquisition means for picking up noise emitted inside one of said modules, and sound generation means for generating counter-noise in said module so as to proceed with canceling said emitted noise and thus reducing the overall noise level of the mail handling machine.

### FIELD OF THE INVENTION

The present invention relates exclusively to the field of mail handling and it relates more particularly to a mail handling machine that has a noise level that is significantly lower than the noise levels of currently known machines.

### PRIOR ART

Today, mail handling machines, in particular postage meters or “franking machines”, feeders, and folding and insertion machines are relatively noisy, the noise levels generated by such machines rarely being lower than 60 dB and often even being close to 80 dB, which is somewhat unpleasant for users when the machines operate for prolonged periods.

In order to reduce such noise levels, numerous solutions exist that essentially use mechanical elements. Such mechanical elements can be covers, vibration dampers, or specific noise-reduction coatings. Such solutions suffer from the drawback of being static, i.e. of being defined in response to a given noise structure, and thus of being poorly adapted to noise of frequency that is actually variable and, in addition, that is spread over a very wide frequency range.

### OBJECT AND DEFINITION OF THE INVENTION

An object of the present invention is to provide a mail handling machine that mitigates the drawbacks of the prior solutions in particular by being adapted to reducing non-constant noise, such as the noise that can be generated by envelopes hitting the conveyor rollers of a mail handling machine.

This object is achieved by a mail handling machine comprising a succession of modules disposed along a conveyor path for conveying mail items from a feed module for feeding in mail items to a collection module for collecting mail items that have been franked by passing through a franking module for printing postage imprints on said mail items, said mail handling machine further comprising at least one sound acquisition means for picking up noise emitted inside one of said modules, and sound generation means for generating counter-noise in said module so as to proceed with canceling said emitted noise and thus reducing the overall noise level of the mail handling machine. Preferably, said sound acquisition means and said sound generation means are disposed in each of the modules of the mail handling machine that have covers.

Thus, with these dynamic rather than static means for reducing noise level, it is possible to adapt in real time to the noise generated by the mail handling machine while said machine is operating.

Advantageously, said sound generation means comprise an actuator stuck to a cover of said module in a manner such as to transform said cover into a loudspeaker, said actuator being controlled by processor means generating said counter-noise.

Preferably, said processor means comprise an adaptive filter means for adapting the characteristics of said counter-noise to the characteristics of said emitted noise in real time.

### BRIEF DESCRIPTION OF THE DRAWINGS

The characteristics and advantages of the present invention appear more clearly from the following description given by way of non-limiting indication, and with reference to the accompanying drawings, in which:

FIG. 1 is a diagrammatic view of a low-noise mail handling machine of the invention;

FIG. 2 shows the various elements involved in reducing the noise level of the machine of FIG. 1; and

FIG. 3 is a flow chart of the various steps of the method enabling the mail handling machine of FIG. 1 to have a low noise level.

### DETAILED DESCRIPTION OF A PREFERRED EMBODIMENT

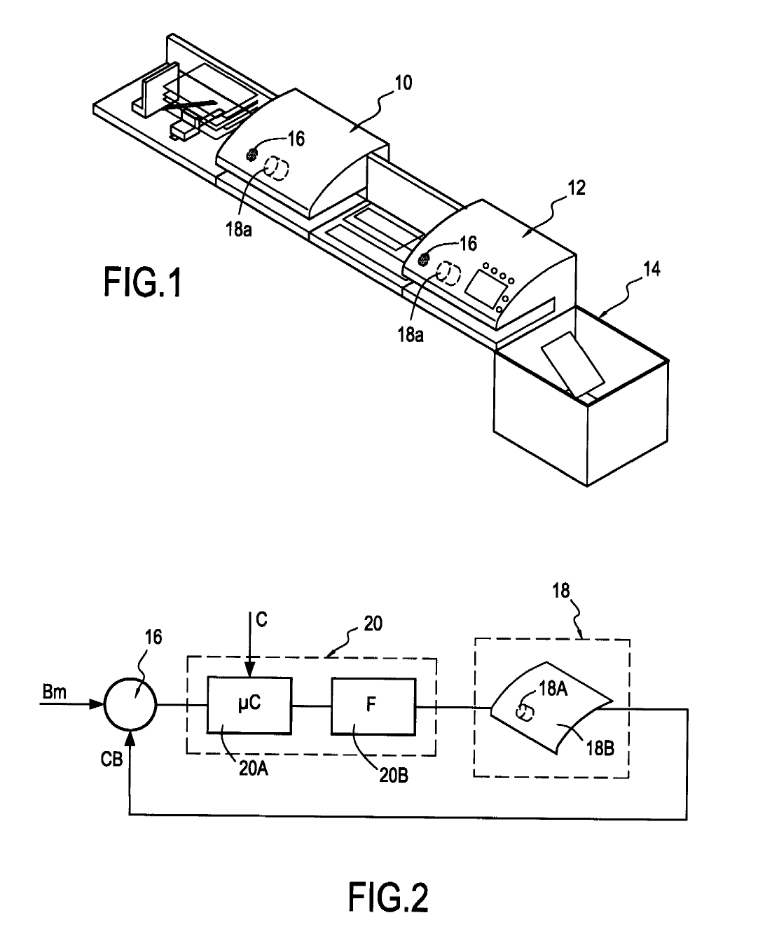

FIG. 1 shows a mail handling machine comprising, in the direction of movement of the mail items, and without the following list being limiting: a feed module 10; a “franking” or postage meter module 12; and a collection module 14. The feed module and the franking module are preferably provided with respective independent devices for actively reducing their noise levels, the object of each device being to generate counter-noise having characteristics that are the inverse of the characteristics of the noise generated by the module while it is operating, so that, once added to the noise, said counter-noise enables said noise to be cancelled.

FIG. 2 shows, more precisely, the various component elements of the structure of each of the noise reducer devices. Each of the noise-reducer devices comprises: sound acquisition means, such as a microphone 16, for picking up the noise existing in a given module of the mail handling machine; sound generation means for generating counter-noise that is the inverse of said noise, which sound generation means comprise a loudspeaker 18; and a processor module 20 for controlling the signal sent to the loudspeaker as a function of the noise picked up by the microphone. The microphone is advantageously disposed as close as possible to the largest noise source of the module in question (e.g. the selection rollers for the franking module). The loudspeaker is advantageously constituted by at least one actuator 18A stuck to the cover 18B of each of the modules. An example of such an actuator is given by the product Soliddrive™ by Induction Dynamics which makes it possible, once stuck to any continuous surface, to transform that surface into a loudspeaker. The processor module conventionally comprises a microprocessor or a Digital Signal Processor (DSP) 20A whose input signal is constituted by the residual noise generated by the microphone 16 and resulting from the difference between the noise Bm emitted by the module and the counter-noise CB generated by the loudspeaker and whose output signal is sent as input to an adaptive filter 20B that is preferably digital and has variable coefficients, and whose output signal controls the loudspeaker. The process is advantageously synchronized to the rate C at which the mail items are handled. U.S. Pat. No. 5,434,925 shows an example of the computations performed by such a processor module in order to cancel the noise generated by a noise source.

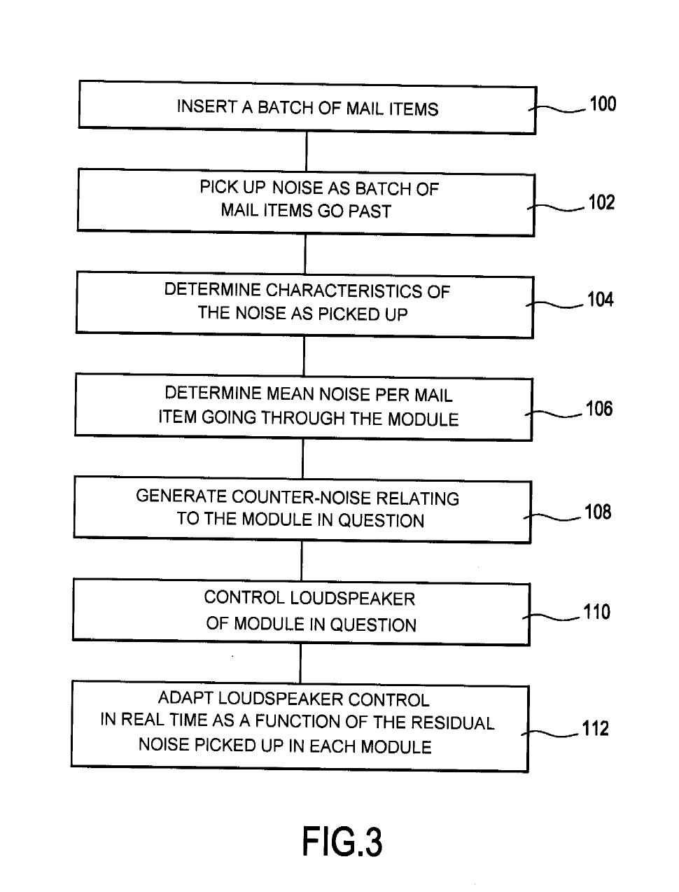

Implementation of reducing sound level at the mail handling machine of the invention is described below with reference to FIG. 3. It firstly assumes that a training stage is performed during which, in step 100, a batch of mail items of identical weight and size, e.g. a batch of about ten envelopes, is inserted into the machine for the purpose of being franked. Said mail items then follow the conveyor path through the machine and pass successively through the various modules making up said machine. In each of the modules, the noise Bm generated by each mail item of the batch to be handled is picked up by the microphone disposed in said module (step 102) making it possible for the processor module to determine, in a step 104, the characteristics of the noises that have been picked up and in particular their periodicities. On the basis of said characteristics, it is thus possible, in step 106, to determine mean noise generated by one envelope passing through a module in question. In step 108, the processor module then computes corresponding counter-noise and controls the loudspeaker accordingly in step 110, the noise as picked up and the counter-noise as generated being synchronized on the basis of the rate at which the mail items are handled, which rate is generally determined by the franking module for franking the mail items.

Once the training stage is finished and while the following mail items are passing through, the microphones of the various modules then pick up only residual noise corresponding to the difference between the noise Bm emitted by the module and the counter-noise CB generated by the loudspeaker, and the processor module endeavors to minimize said residual noise by continuously adapting the coefficients of its digital filter means, thereby performing real-time control of the counter-noise generated by each loudspeaker (step 112).

It should be noted that although the above description mentions only two particular modules of the mail handling machine, the invention naturally applies to any other types of modules having covers and constituting such a machine, e.g. a differential weigh module or a folding or insertion module.

What is claimed is:

1. A mail handling machine comprising a succession of modules disposed along a conveyor path for conveying mail items from a feed module for feeding in mail items to a collection module for collecting mail items that have been franked by passing through a franking module for printing postage imprints on said mail items, said mail handling machine further comprising at least one sound acquisition means for picking up noise emitted inside one of said modules, and sound generation means for generating counter-noise in said module so as to proceed with canceling said emitted noise and thus reducing the overall noise level of the mail handling machine.

2. A mail handling machine according to claim 1, wherein said sound generation means comprise an actuator stuck to a cover of said module in a manner such as to transform said cover into a loudspeaker, said actuator being controlled by processor means generating said counter-noise.

3. A mail handling machine according to claim 2, wherein said processor means comprise an adaptive filter means for adapting the characteristics of said counter-noise to the characteristics of said emitted noise in real time.

4. A mail handling machine according to claim 1, wherein said sound acquisition means and said sound generation means are disposed in each of the modules of the mail handling machine that have covers.
